(19) United States
(12) Patent Application Publication
(10) Pub. No.: US 2026/0144480 A1
(43) Pub. Date: May 28, 2026

(54) IN-VIVO TESTING DEVICE FOR DYNAMICALLY MEASURING THE MECHANICAL PROPERTIES OF SKIN

(71) Applicant: Ran Huang, Shanghai (CN)

(72) Inventor: Ran Huang, Shanghai (CN)

(21) Appl. No.: 19/360,603

(22) Filed: Oct. 16, 2025

(30) Foreign Application Priority Data
Nov. 22, 2024 (CN) 202411676064.3

(51) Int. Cl. A61B 5/00; A61B 90/00

(52) U.S. Cl. CPC A61B 5/442; A61B 5/0048; A61B 90/06; A61B 2090/064

(57) ABSTRACT

An in-vivo testing device is disclosed for dynamically measuring the mechanical properties of skin. The device includes a horizontal reciprocating tensile stress-strain detection unit configured to contact the skin surface and acquire displacement and tensile force data, wherein the detection unit comprises at least two contact heads. A driving unit, equipped with a motor capable of outputting reciprocating linear motion perpendicular to the skin surface, is provided to drive the contact heads. A reversing unit converts the perpendicular reciprocating linear motion into a horizontal reciprocating tensile motion of the contact heads relative to the skin surface. The reversing unit includes at least one right-angle reversing lever connecting the driving unit and the detection unit. The disclosed device enables reliable evaluation of the dynamic viscoelastic properties of skin in a non-destructive manner, thereby providing accurate and repeatable characterization of skin biomechanics under in-vivo conditions.

### TECHNICAL FIELD

The present invention relates to in-vivo testing devices, and more particularly to an in-vivo device for dynamically measuring the mechanical properties of skin materials.

### BACKGROUND

The mechanical properties of skin can be measured using various techniques, which are generally divided into ex-vivo tests and in-vivo tests.

In ex-vivo experiments, the skin sample is usually excised from a living body, or replaced by artificial skin made from animal tissue, gelatin, or polyurethane. The mechanical properties measured in such cases differ from those of skin in its natural state and may raise ethical concerns. In contrast, in-vivo experiments are non-destructive, which ensures the reliability, repeatability, and accessibility of testing. Methods such as indentation, torsion, stretching, suction, dynamic testing, and optical techniques have all been employed to characterize the mechanical properties of skin.

In classical materials science, viscoelasticity is typically characterized using dynamic mechanical analysis (DMA), in which a small cyclic strain is applied to a sample and the resulting stress response is measured. Therefore, when applying this principle to living tissues, direct measurement of cyclic and continuous responses is indispensable. Existing skin mechanical testing devices mainly focus on measuring skin elasticity and mechanical properties under quasi-static strain rates, and have not been specifically designed to evaluate the dynamic characteristics of skin.

Moreover, existing in-vivo testing devices are often bulky, making them inconvenient for portability and practical use.

### SUMMARY

The present invention has been made to overcome the foregoing problems, and its objective is to provide an in-vivo device for dynamically measuring the mechanical properties of skin materials.

According to one aspect of the invention, there is provided an in-vivo testing device for dynamically measuring the mechanical properties of skin, comprising: a horizontal reciprocating tensile stress-strain detection unit configured to contact the skin surface under test and to measure displacement data and tensile force data, the detection unit including at least two contact heads; a driving unit configured to drive the contact heads, the driving unit comprising a driving motor that outputs a reciprocating linear motion perpendicular to the skin surface under test; and a reversing unit configured to convert the reciprocating linear motion into a horizontal reciprocating tensile motion of the contact heads relative to the skin surface, the reversing unit including at least one right-angle reversing lever configured to connect the driving unit and the horizontal reciprocating tensile stress-strain detection unit.

In some embodiments, the driving motor includes an output shaft that reciprocates along a first direction. The right-angle reversing lever is formed of a first rod portion and a second rod portion that are perpendicular to each other and joined at their ends. An end of the first rod portion remote from the second rod portion is rotatably connected to the end of the output shaft. The reversing unit further includes: a first steering limiting slide rail extending along a second direction forming an angle with the first direction; a first steering limiting slider slidably disposed on the first steering limiting slide rail and rotatably connected to the junction of the first and second rod portions; a second steering limiting slide rail extending along a third direction perpendicular to the first direction; and a second steering limiting slider slidably disposed on the second steering limiting slide rail and rotatably connected to the end of the second rod portion remote from the first rod portion.

In some embodiments, the reversing unit further includes a first rotary connection assembly configured to connect the second steering limiting slider to the end of the second rod portion remote from the first rod portion. The number of contact heads is two. The horizontal reciprocating tensile stress-strain detection unit further comprises: a detection slider slidably disposed on the second steering limiting slide rail; a contact head mounting plate for mounting the two contact heads arranged along the first direction, wherein the contact heads are parallel and located on the same horizontal plane; a tensile force sensor for detecting tensile force data generated along the third direction; a detection connecting member fixed on the detection slider and configured to connect the contact head mounting plate with one end of the tensile force sensor; a displacement sensor for detecting displacement data, the housing of which is arranged along the third direction; and a displacement sensor mounting frame disposed adjacent to the detection connecting member for fixing the housing, wherein the other end of the tensile force sensor is fixedly connected to the second steering limiting slider, and the movable core of the displacement sensor is connected to the detection connecting member.

In some embodiments, the driving motor includes an output shaft that reciprocates along the first direction, and the reversing unit comprises two right-angle reversing levers, denoted as a first and a second right-angle reversing lever. Each reversing lever comprises a first rod portion and a second rod portion that are perpendicular and connected at their ends. The ends of the first rod portions of the two reversing levers remote from the respective second rod portions are rotatably connected to the output shaft. The reversing unit further includes: a first steering limiting slide rail extending along a second direction forming an angle with the first direction; a first steering limiting slider slidably disposed on the first steering limiting slide rail and rotatably connected to the junction of the first and second rod portions of the first reversing lever; a second steering limiting slide rail extending along a third direction perpendicular to the first direction; a second steering limiting slider slidably disposed on the second steering limiting slide rail and rotatably connected to the end of the second rod portion of the first reversing lever; a third steering limiting slide rail extending along a fourth direction forming said angle with the first direction; a third steering limiting slider slidably disposed on the third steering limiting slide rail and rotatably connected to the junction of the first and second rod portions of the second reversing lever; and a fourth steering limiting slider slidably disposed on the second steering limiting slide rail and rotatably connected to the end of the second rod portion of the second reversing lever.

In some embodiments, the reversing unit further comprises a first rotary connection assembly connecting the second steering limiting slider with the end of the second rod portion of the first right-angle reversing lever, and a second rotary connection assembly connecting the fourth steering limiting slider with the end of the second rod portion of the second right-angle reversing lever. The horizontal reciprocating tensile stress-strain detection unit comprises two contact heads, denoted as a first and a second contact head, and further includes: a first detection slider slidably disposed on the second steering limiting slide rail; a first contact head mounting plate configured to mount the first contact head along the first direction; a first tensile force sensor for detecting tensile force data generated by the first contact head along the third direction; a first detection connecting member fixed on the first detection slider and configured to connect the first contact head mounting plate with one end of the first tensile force sensor; a first displacement sensor for detecting displacement data of the first contact head, the housing of which is arranged along the third direction; a first displacement sensor mounting frame for fixing the housing of the first displacement sensor; a second detection slider slidably disposed on the second steering limiting slide rail; a second contact head mounting plate configured to mount the second contact head along the first direction; a second tensile force sensor for detecting tensile force data generated by the second contact head along the third direction; a second detection connecting member fixed on the second detection slider and configured to connect the second contact head mounting plate with one end of the second tensile force sensor; a second displacement sensor for detecting displacement data of the second contact head, the housing of which is arranged along the third direction; and a second displacement sensor mounting frame for fixing the housing of the second displacement sensor,
wherein the other end of the first tensile force sensor is fixedly connected with the second steering limiting slider, the movable core of the first displacement sensor is connected with the first detection connecting member, the other end of the second tensile force sensor is fixedly connected with the fourth steering limiting slider, and the movable core of the second displacement sensor is connected with the second detection connecting member, with the first and second contact heads being parallel and positioned on the same horizontal plane.

In some embodiments, the reversing unit further includes a first connection plate fixedly connected to the end of the first rod portion of the first right-angle reversing lever remote from the second rod portion, and a second connection plate fixedly connected to the end of the first rod portion of the second right-angle reversing lever remote from the second rod portion. The driving unit further includes a driving connection assembly, comprising a connecting shaft and, sequentially mounted thereon, a first fastener, a first driving connector, a first positioning shaft sleeve, a first bearing cover plate, a first bearing, a shaft collar, a second bearing, a second bearing cover plate, a second positioning shaft sleeve, a second driving connector, and a second fastener. The first connection plate is provided with a through hole for embedding the first bearing, and the second connection plate is provided with a through hole for embedding the second bearing. One end of the first driving connector is provided with a through hole for mounting on the connecting shaft, and the other end is connected to the output shaft. One end of the second driving connector is also provided with a through hole for mounting on the connecting shaft, and the other end is connected to the output shaft.

In some embodiments, the driving unit further includes: a driving limiting slide rail extending along the first direction and disposed below the output shaft; a driving limiting slider slidably disposed on the driving limiting slide rail; an L-shaped driving connection plate fixed on the driving limiting slider and configured to connect the output shaft with the other ends of the first and second driving connectors; and a fixed bracket fixedly connected to the bottom of the housing of the driving motor.

In some embodiments, the first fastener and the second fastener are locking nuts, double nuts, or, based on a single nut, further secured by a double-ear lock washer, a Nord-Lock washer, or a thread locking adhesive.

In some embodiments, the vertical effective leg lengths of the first rod portion and the second rod portion of the right-angle reversing lever are preset lengths, respectively, such that, through the preset lengths and the included angle, the displacement of the contact heads has a fixed geometric relationship with the displacement of the output shaft.

In some embodiments, the included angle is 45 degrees, and the preset lengths of the first rod portion and the second rod portion are equal, such that the displacement of the contact heads equals the displacement of the output shaft.

### Advantages and Effects of the Invention

According to the in-vivo device for dynamically measuring the mechanical properties of skin materials disclosed in the present invention, the vertical reciprocating linear motion of the driving motor, which is perpendicular to the skin surface under test, is converted into a horizontal reciprocating tensile motion of the contact heads relative to the skin surface by means of a right-angle reversing lever. This arrangement enables direct measurement of the mechanical properties of skin materials.

Therefore, the device of the present invention can be effectively used to evaluate the dynamic characteristics of skin.

### BRIEF DESCRIPTION OF DRAWINGS

FIG. 1 is a schematic structural diagram of an in-vivo device for dynamically measuring the mechanical properties of skin according to an embodiment of the present invention.

FIG. 2 is a schematic structural diagram of a driving unit according to an embodiment of the present invention.

FIG. 3 is an exploded view of a driving connection assembly according to an embodiment of the present invention.

FIG. 4 is a schematic structural diagram of a reversing unit according to an embodiment of the present invention.

FIG. 5 is an exploded view of a first limiting connection assembly according to an embodiment of the present invention.

FIG. 6 is an exploded view of a first rotary connection assembly according to an embodiment of the present invention.

FIG. 7 is a schematic structural diagram of a horizontal reciprocating tensile stress-strain detection unit according to an embodiment of the present invention.

### DETAILED DESCRIPTION OF EMBODIMENTS

To make the technical means, inventive features, objectives, and effects of the present invention more readily understandable, the following embodiments are described in detail with reference to the accompanying drawings.

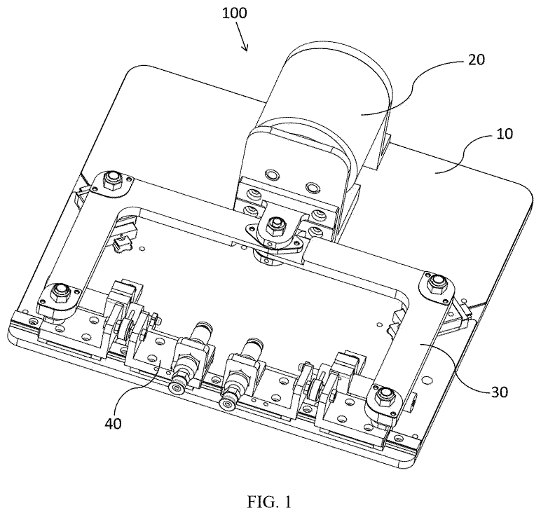

FIG. 1 illustrates a schematic structural diagram of an in-vivo device 100 for dynamically measuring the mechanical properties of skin according to an embodiment of the present invention.

As shown in FIG. 1, the device 100 comprises a base plate 10, a driving unit 20, a reversing unit 30, and a horizontal reciprocating tensile stress-strain detection unit 40.

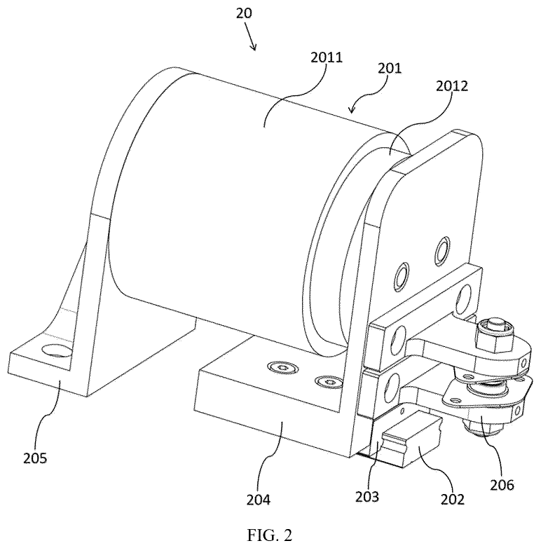

FIG. 2 illustrates a schematic structural diagram of the driving unit according to an embodiment of the present invention.

As shown in FIG. 2, the driving unit 20 includes a driving motor 201, a driving limiting slide rail 202, a driving limiting slider 203, an L-shaped driving connection plate 204, a fixed bracket 205, and a driving connection assembly 206.

The driving motor 201 includes a housing 2011 and an output shaft 2012, which performs reciprocating linear motion along a first direction, i.e., perpendicular to the skin surface under test. In this embodiment, the driving motor 201 is a voice coil motor, and the displacement amplitude of the output shaft 2012 in its forward and backward motion ranges within ±5 mm, with a cycle frequency not exceeding 100 Hz.

The driving limiting slide rail 202 extends along the first direction and is disposed below the output shaft 2012. It is fixed to the base plate 10. In this embodiment, positioning grooves are pre-formed on the base plate 10 for placement of the driving limiting slide rail 202, and threaded screws are used to fix the slide rail 202 onto the base plate 10.

The driving limiting slider 203 is slidably mounted on the driving limiting slide rail 202.

One side of the L-shaped driving connection plate 204 is fixed to the driving limiting slider 203, and the other side is fixed to the output shaft 2012. In this embodiment, threaded holes are formed on both sides of the L-shaped driving connection plate 204, and screws are used to fasten it to the output shaft 2012 and the driving limiting slider 203, respectively. With the driving limiting slide rail 202, driving limiting slider 203, and L-shaped driving connection plate 204, the output trajectory of the output shaft 2012 is corrected, ensuring precise reciprocating linear motion strictly along the first direction.

The fixed bracket 205 is mounted on the base plate 10 and fixed to the bottom of the housing 2011.

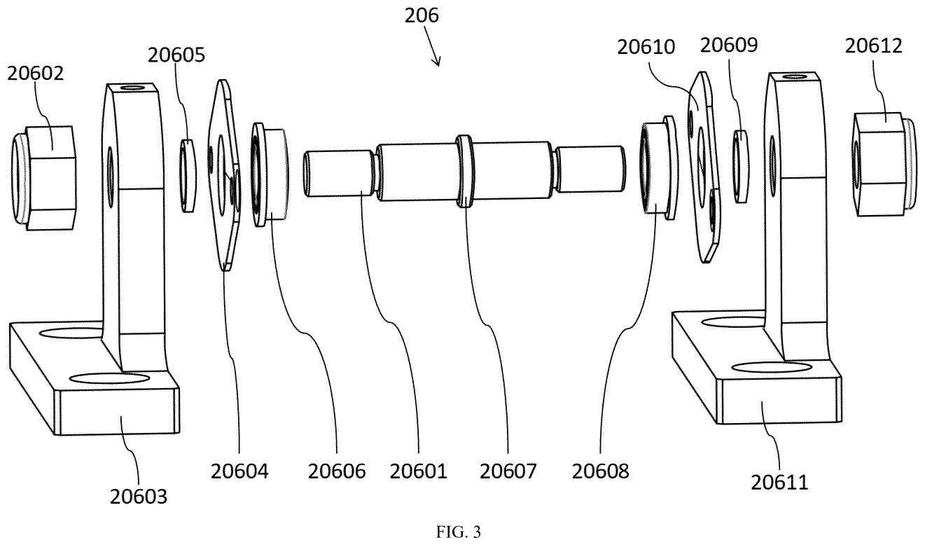

FIG. 3 illustrates an exploded view of a driving connection assembly according to an embodiment of the present invention.

As shown in FIG. 3, the driving connection assembly 206 comprises a connecting shaft 20601 and, sequentially mounted on the connecting shaft 20601, a first fastener 20602, a first driving connector 20603, a first positioning shaft sleeve 20605, a first bearing cover plate 20604, a first bearing 20606, a shaft collar 20607, a second bearing 20608, a second bearing cover plate 20610, a second positioning shaft sleeve 20609, a second driving connector 20611, and a second fastener 20612. In this embodiment, the shaft collar 20607 is integrally formed with the connecting shaft 20601.

One end of the first driving connector 20603 is provided with a through hole for mounting on the connecting shaft 20601, and the other end is fixedly connected to the L-shaped driving connection plate 204. One end of the second driving connector 20611 is likewise provided with a through hole for mounting on the connecting shaft 20601, and the other end is fixedly connected to the L-shaped driving connection plate 204.

The first fastener 20602 and the second fastener 20612 may be locking nuts, double nuts, or single nuts additionally secured by a double-ear lock washer, a Nord-Lock washer, or a thread locking adhesive.

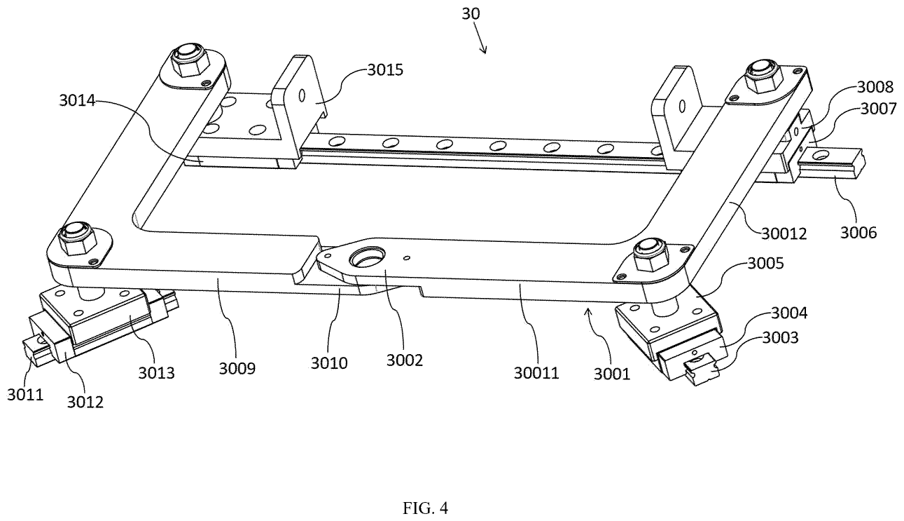

FIG. 4 illustrates a schematic structural diagram of the reversing unit according to an embodiment of the present invention.

As shown in FIG. 4, the reversing unit 30 comprises a first right-angle reversing lever 3001, a first connection plate 3002, a first steering limiting slide rail 3003, a first steering limiting slider 3004, a first limiting connection assembly 3005, a second steering limiting slide rail 3006, a second steering limiting slider 3007, a first rotary connection assembly 3008, a second right-angle reversing lever 3009, a second connection plate 3010, a third steering limiting slide rail 3011, a third steering limiting slider 3012, a second limiting connection assembly 3013, a fourth steering limiting slider 3014, and a second rotary connection assembly 3015. In this embodiment, the first right-angle reversing lever 3001 and the second right-angle reversing lever 3009 have the same thickness and are positioned at the same height level, thereby minimizing possible data errors.

The first right-angle reversing lever 3001 is formed by a first rod portion 30011 and a second rod portion 30012, which are perpendicular and connected at their ends. In this embodiment, the first rod portion 30011 and the second rod portion 30012 are integrally formed to constitute the first right-angle reversing lever 3001.

One end of the first connection plate 3002 is fixedly connected to the end of the first rod portion 30011 of the first right-angle reversing lever 3001 remote from the second rod portion 30012. The other end of the first connection plate 3002 is provided with a through hole for embedding the first bearing 20606, such that the first connection plate 3002 is rotatably connected to the output shaft 2012. In this embodiment, the upper surface of the first connection plate 3002 is flush with the upper surface of the first rod portion 30011, and the thickness of the first connection plate 3002 is one-half of that of the first rod portion 30011. The first bearing 20606 is a flange bearing, and the two ends of its bearing ring have different diameters. Therefore, the through hole on the first rod portion is a stepped hole matching the corresponding bearing diameters.

In this embodiment, when the bearing ring on the larger-diameter side of the first bearing 20606 is embedded in the first connection plate 3002, the ring protrudes slightly above the surface of the plate. The first bearing cover plate 20604 is provided with a through hole, the size of which allows the first positioning shaft sleeve 20605 to pass through but not the bearing ring of the first bearing 20606. Accordingly, screws are used to fasten the first bearing cover plate 20604 to the first connection plate 3002, thereby fixing the first bearing 20606 in place. The first bearing cover plate 20604 is diamond-shaped so that the fastening screws do not interfere with moving parts. In this embodiment, similar configurations of shaft sleeves, bearing cover plates, and stepped holes are adopted for all rotary connection structures that employ bearings, and will not be repeated. The shape of the bearing cover plate may be adaptively adjusted according to the trajectory of moving parts to avoid interference.

The first steering limiting slide rail 3003 extends along a second direction forming an angle with the first direction, and is fixed to the base plate 10. In this embodiment, the preferred angle range is 44.5-45.5 degrees. Positioning grooves are pre-formed on the base plate 10 for placement of the first steering limiting slide rail 3003, and threaded screws are used to secure it to the base plate 10.

The first steering limiting slider 3004 is slidably mounted on the first steering limiting slide rail 3003.

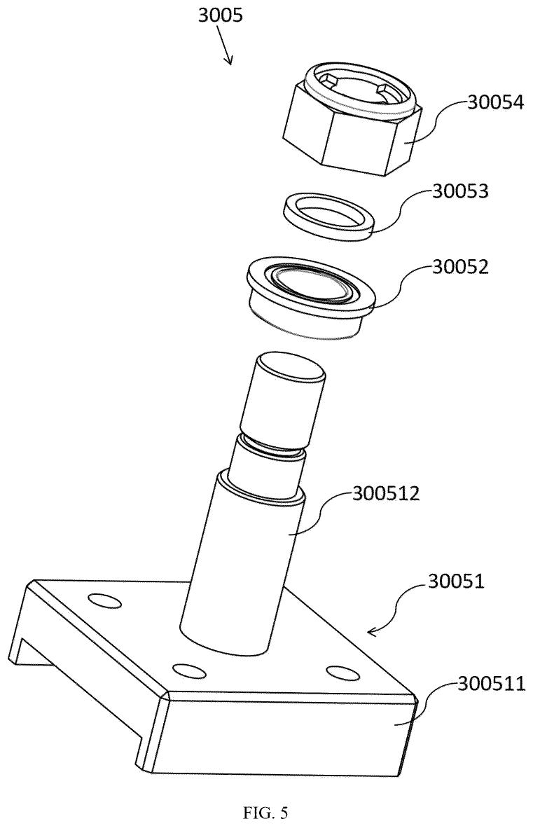

FIG. 5 illustrates an exploded view of a first limiting connection assembly according to an embodiment of the present invention.

As shown in FIG. 5, the first limiting connection assembly 3005 comprises a first connecting base 30051, a third bearing 30052, a third bearing cover plate (not shown), a third positioning shaft sleeve 30053, and a third fastener 30054.

The first connecting base 30051 includes a base portion 300511 fixedly connected to the first steering limiting slider 3004, and a column portion 300512 fixedly provided on one end of the base portion 300511. In this embodiment, the base portion 300511 and the column portion 300512 are integrally formed to constitute the first connecting base 30051.

The third positioning shaft sleeve 30053 is inserted into a through hole located at the junction of the first rod portion 30011 and the second rod portion 30012, such that the third positioning shaft sleeve 30053 is disposed at the right angle of the first right-angle reversing lever 3001.

The third bearing 30052, the third bearing cover plate, the third positioning shaft sleeve 30053, and the third fastener 30054 are sequentially mounted on the column portion 300512. The upper diameter of the column portion 300512 is smaller than its lower diameter, and the inner diameter of the third bearing 30052 is smaller than the lower diameter of the column portion 300512, while the lower outer diameter of the third bearing 30052 is larger than the lower diameter of the column portion 300512. Thus, the first right-angle reversing lever 3001 is fixed at a predetermined height and rotatably connected with the first steering limiting slider 3004.

The second steering limiting slide rail 3006 extends along a third direction perpendicular to the first direction and is fixedly mounted on the base plate 10. Positioning grooves are pre-formed on the base plate 10 for placement of the second steering limiting slide rail 3006, and threaded screws are used to secure the slide rail 3006 to the base plate 10.

The second steering limiting slider 3007 is slidably mounted on the second steering limiting slide rail 3006.

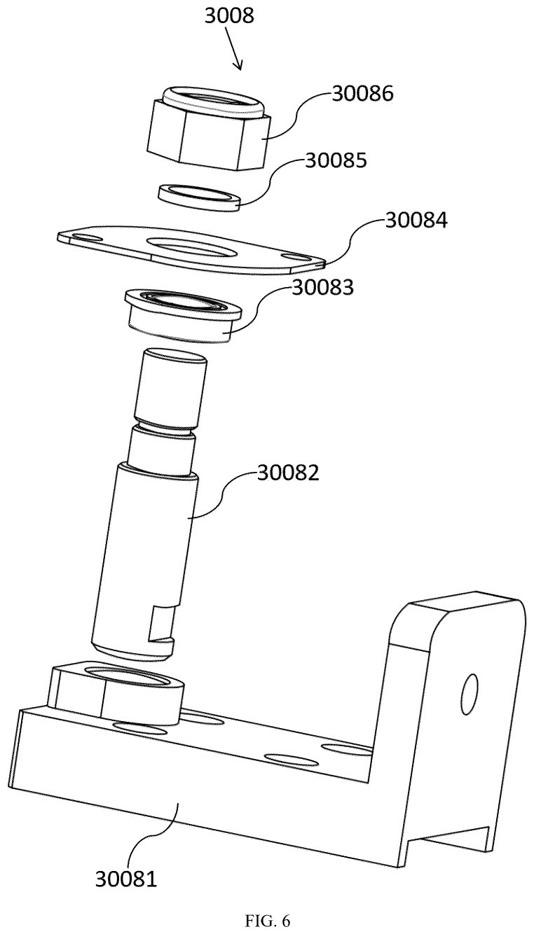

FIG. 6 illustrates an exploded view of a first rotary connection assembly according to an embodiment of the present invention.

As shown in FIG. 6, the first rotary connection assembly 3008 comprises a second connecting base 30081, a connecting post 30082, a fourth bearing 30083, a fourth bearing cover plate 30084, a fourth positioning shaft sleeve 30085, and a fourth fastener 30086.

The second connecting base 30081 is fixedly connected to the second steering limiting slider 3007. The second connecting base 30081 is L-shaped, with one side provided with a hole for insertion of the connecting post 30082.

The upper diameter of the connecting post 30082 is smaller than its lower diameter, and the lower portion is provided with a groove. In this embodiment, the side of the second connecting base 30081 provided with the insertion hole is also provided with a threaded fixing hole. After the connecting post 30082 is inserted, a bolt passes through the fixing hole and engages with the groove, thereby securing the connecting post 30082 to the second connecting base 30081.

The fourth bearing 30083, the fourth bearing cover plate 30084, the fourth positioning shaft sleeve 30085, and the fourth fastener 30086 are sequentially mounted on the connecting post 30082. The inner diameter of the fourth bearing 30083 is smaller than the lower diameter of the connecting post 30082, and the lower outer diameter of the fourth bearing 30083 is larger than the lower diameter of the connecting post 30082. As a result, the first right-angle reversing lever 3001 is fixed at a predetermined height and rotatably connected with the second steering limiting slider 3007. In this embodiment, the relative height at the junction of the upper and lower sections of the column portion 300512 and the connecting post 30082 is adjusted such that the entire first right-angle reversing lever 3001 remains in the same horizontal plane.

The second connection plate 3010 is fixedly connected to the end of the second right-angle reversing lever 3009 near the driving unit 20. In this embodiment, the lower surface of the second connection plate 3010 is flush with the lower surface of the second right-angle reversing lever 3009, and the thickness of the second connection plate 3010 is half the thickness of the second right-angle reversing lever 3009. When the first and second right-angle reversing levers 3001, 3009 are connected to the driving connection assembly 206, both reversing levers are positioned in the same horizontal plane.

The second right-angle reversing lever 3009, the third steering limiting slide rail 3011, the third steering limiting slider 3012, the second limiting connection assembly 3013, the fourth steering limiting slider 3014, and the second rotary connection assembly 3015 correspond respectively to the first right-angle reversing lever 3001, the first steering limiting slide rail 3003, the first steering limiting slider 3004, the first limiting connection assembly 3005, the second steering limiting slider 3007, and the first rotary connection assembly 3008, arranged in a mirror-symmetrical configuration along the first direction. The specific structures will not be repeated.

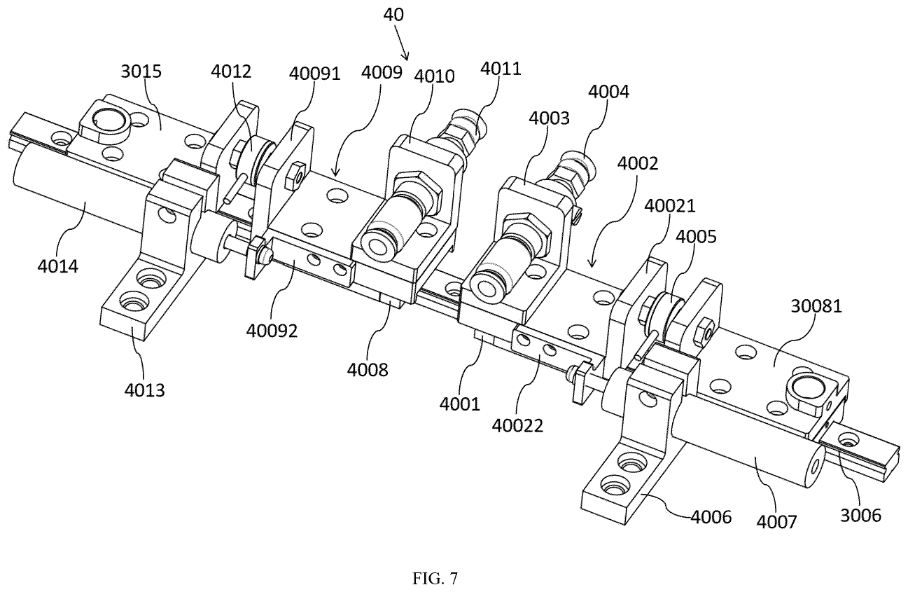

FIG. 7 illustrates a schematic structural diagram of the horizontal reciprocating tensile stress-strain detection unit according to an embodiment of the present invention.

As shown in FIG. 7, the horizontal reciprocating tensile stress-strain detection unit 40 comprises a first detection slider 4001, a first detection connecting member 4002, a first contact head mounting plate 4003, a first contact head 4004, a first tensile force sensor 4005, a first displacement sensor mounting frame 4006, a first displacement sensor 4007, a second detection slider 4008, a second detection connecting member 4009, a second contact head mounting plate 4010, a second contact head 4011, a second tensile force sensor 4012, a second displacement sensor mounting frame 4013, and a second displacement sensor 4014.

The first detection slider 4001 is slidably disposed on the second steering limiting slide rail 3006.

The first detection connecting member 4002 comprises a first detection sub-member 40021 and a second detection sub-member 40022, both of which are L-shaped plates composed of two perpendicular straight portions. One straight portion of the first detection sub-member 40021 is fixedly mounted on the first detection slider 4001, and the side surface of this straight portion is fixedly connected to one straight portion of the second detection sub-member 40022.

The first contact head mounting plate 4003 is also an L-shaped plate composed of two perpendicular straight portions. One straight portion of the first contact head mounting plate 4003 is fixedly connected to the straight portion of the first detection sub-member 40021 mounted on the first detection slider 4001. The other straight portion is provided with a through hole, through which the first contact head 4004 is mounted and fixed along the first direction.

One end of the first tensile force sensor 4005 passes through a through hole in the straight portion of the first detection sub-member 40021 and is fastened thereto by screws, while the other end of the sensor 4005 is fastened to a through hole located on the side of the second connecting base 30081 remote from the connecting post 30082. The straight portion of the first detection sub-member 40021 is not fixedly connected to the first detection slider 4001. The first tensile force sensor 4005 is configured to detect tensile force data generated by the first contact head 4004 along the third direction.

The first displacement sensor mounting frame 4006 is disposed adjacent to the first detection connecting member 4002 and is fixedly mounted to the base plate 10. It is configured to fix the housing of the first displacement sensor 4007, which is oriented along the third direction.

The first displacement sensor 4007 is configured to detect displacement data of the first contact head 4004. Its movable core is secured, via a through hole, to a straight portion of the second detection sub-member 40022 by screws. This straight portion of the second detection sub-member 40022 is not connected to the first detection sub-member 40021.

The second detection slider 4008 is slidably disposed on the second steering limiting slide rail 3006.

The second detection connecting member 4009 comprises a third detection sub-member 40091 and a fourth detection sub-member 40092, both of which are L-shaped plates composed of two perpendicular straight portions. One straight portion of the third detection sub-member 40091 is fixedly mounted on the second detection slider 4008, and the side surface of this straight portion is fixedly connected to one straight portion of the fourth detection sub-member 40092.

The second contact head mounting plate 4010 is also an L-shaped plate composed of two perpendicular straight portions. One straight portion of the second contact head mounting plate 4010 is fixedly connected to the straight portion of the third detection sub-member 40091 mounted on the second detection slider 4008. The other straight portion is provided with a through hole, through which the second contact head 4011 is mounted and fixed along the first direction.

One end of the second tensile force sensor 4012 passes through a through hole in the straight portion of the third detection sub-member 40091 and is fastened thereto by screws, while the other end of the sensor 4012 is fastened to a through hole of the second rotary connection assembly 3015. The straight portion of the third detection sub-member 40091 is not fixedly connected to the second detection slider 4008. The second tensile force sensor 4012 is configured to detect tensile force data generated by the second contact head 4011 along the third direction. In this embodiment, the screw plane at the bottom of the rods of the first tensile force sensor 4005 and the second tensile force sensor 4012, close to the welded joint, is in direct contact with adjacent parts, while the plane at the opposite side is provided with a clearance gap, avoiding direct contact.

The second displacement sensor mounting frame 4013 is disposed adjacent to the second detection connecting member 4009 and is fixedly mounted to the base plate 10. It is configured to fix the housing of the second displacement sensor 4014, which is oriented along the third direction.

The second displacement sensor 4014 is configured to detect displacement data of the second contact head 4011. Its movable core is secured, via a through hole, to a straight portion of the fourth detection sub-member 40092 by screws. This straight portion of the fourth detection sub-member 40092 is not connected to the third detection sub-member 40091. In this embodiment, since the displacement of the first contact head 4004 and the second contact head 4011 is identical, only one displacement sensor (either the second displacement sensor 4014 or the first displacement sensor 4007) may be retained as needed. The displacement value acquired by the retained sensor can represent the displacement of either contact head.

The first contact head 4004 and the second contact head 4011 are arranged parallel to each other and are positioned on the same horizontal plane. In this embodiment, both contact heads 4004 and 4011 are vacuum suction cups, each having a suction head with a diameter of 8 mm. The initial distance between the centers of the suction heads of the two contact heads is 25 mm.

The vertical effective leg lengths of the two rod portions of the first and second right-angle reversing levers 3001 and 3009 are defined as preset lengths, respectively. Through the preset lengths and the included angle, the displacement of the first contact head 4004 and the second contact head 4011 has a fixed geometric relationship with the displacement of the output shaft 2012. For example, in this embodiment, the displacement of both contact heads 4004 and 4011 is approximately equal to the displacement of the output shaft 2012. When the included angle is set to 45 degrees, and both preset lengths are equal (8 cm), the displacement of the contact heads 4004 and 4011 is approximately identical to that of the output shaft 2012. Furthermore, under the 45-degree configuration, the angle between the second rod portion 30012 and the second steering limiting slide rail 3006 during motion ranges between 86.52° and 93.7°. With these design parameters, the overall length and width of the device 100 are 20 cm, and the height is 8 cm, thereby effectively achieving portability.

In use, the device 100 for dynamically measuring the mechanical properties of skin materials is operated as follows.

Measurement preparation stage: The tested skin region is first cleaned and dried. Then, the two contact heads 4004 and 4011 are positioned on the surface of the skin to be measured. The suction heads are made to contact the skin surface, and the vacuum system is activated such that the suction cups firmly attach to the skin. This ensures that the contact heads are securely coupled to the skin surface, thereby avoiding slippage during testing.

Measurement stage: The driving motor 201 outputs a reciprocating linear motion along the first direction (i.e., perpendicular to the skin surface under test). Through the reversing unit 30, the motion of the output shaft 2012 is converted into horizontal reciprocating tensile motion of the two contact heads 4004 and 4011 along the third direction (i.e., parallel to the skin surface). As the contact heads 4004 and 4011 perform horizontal reciprocating tensile motion, the tensile force sensors 4005 and 4012 detect the tensile force data of the skin, and the displacement sensors 4007 and 4014 detect the displacement data of the skin.

During this process, the horizontal reciprocating displacement values of the two contact heads 4004 and 4011 are substantially identical. Therefore, the device can output reliable tensile stress-strain data of the skin by processing and analyzing the tensile force and displacement signals collected by the sensors.

Through the above configuration and measurement method, the device 100 can conveniently and effectively measure the dynamic mechanical properties of skin in vivo. The device is compact, portable, and allows non-destructive, repeatable, and reliable testing.

What is claimed is:

1. An in-vivo testing device for dynamically measuring the mechanical properties of skin, comprising:
a horizontal reciprocating tensile stress-strain detection unit configured to contact the skin surface under test and to detect displacement data and tensile force data, the detection unit comprising at least two contact heads, a tensile force sensor, and a displacement sensor;
a driving unit configured to drive the contact heads, the driving unit including a driving motor that outputs a reciprocating linear motion perpendicular to the skin surface under test, the driving motor having an output shaft that moves reciprocally along a first direction; and
a reversing unit configured to convert the reciprocating linear motion into a horizontal reciprocating tensile motion of the contact heads relative to the skin surface, the reversing unit comprising at least one right-angle reversing lever formed by a first rod portion and a second rod portion connected perpendicularly at their ends, the right-angle reversing lever being configured to connect the driving unit and the horizontal reciprocating tensile stress-strain detection unit.

2. The device of claim 1, wherein an end of the first rod portion remote from the second rod portion is rotatably connected to the end of the output shaft, and the reversing unit further comprises:
a first steering limiting slide rail extending along a second direction forming an angle with the first direction;
a first steering limiting slider slidably disposed on the first steering limiting slide rail and rotatably connected to the junction of the first and second rod portions;
a second steering limiting slide rail extending along a third direction perpendicular to the first direction; and
a second steering limiting slider slidably disposed on the second steering limiting slide rail and rotatably connected to the end of the second rod portion remote from the first rod portion.

3. The device of claim 2, wherein the reversing unit further comprises a first rotary connection assembly connecting the second steering limiting slider with the end of the second rod portion remote from the first rod portion, and wherein the number of contact heads is two, the horizontal reciprocating tensile stress-strain detection unit further comprising:
a detection slider slidably disposed on the second steering limiting slide rail;
a contact head mounting plate for mounting the two contact heads arranged along the first direction, the contact heads being parallel and located at the same height;
a tensile force sensor for detecting tensile force data generated along the third direction;
a detection connecting member fixed on the detection slider, configured to connect the contact head mounting plate with one end of the tensile force sensor;
a displacement sensor for detecting displacement data, the housing of which is disposed along the third direction; and
a displacement sensor mounting frame arranged beside the detection connecting member and configured to fix the housing,
wherein the other end of the tensile force sensor is fixedly connected with the second steering limiting slider, and a movable core of the displacement sensor is connected with the detection connecting member.

4. The device of claim 1, wherein the reversing unit comprises two right-angle reversing levers, denoted as a first right-angle reversing lever and a second right-angle reversing lever, and wherein ends of the first rod portions of the first and second right-angle reversing levers remote from the respective second rod portions are rotatably connected to the output shaft, the reversing unit further comprising:
a first steering limiting slide rail extending along a second direction forming an angle with the first direction;
a first steering limiting slider slidably disposed on the first steering limiting slide rail and rotatably connected to the junction of the first and second rod portions of the first right-angle reversing lever;
a second steering limiting slide rail extending along a third direction perpendicular to the first direction;
a second steering limiting slider slidably disposed on the second steering limiting slide rail and rotatably connected to the end of the second rod portion of the first right-angle reversing lever remote from the first rod portion;
a third steering limiting slide rail extending along a fourth direction forming said angle with the first direction;
a third steering limiting slider slidably disposed on the third steering limiting slide rail and rotatably connected to the junction of the first and second rod portions of the second right-angle reversing lever; and a fourth steering limiting slider slidably disposed on the second steering limiting slide rail and rotatably connected to the end of the second rod portion of the second right-angle reversing lever remote from the first rod portion.

5. The device of claim 4, wherein the reversing unit further comprises a first rotary connection assembly connecting the second steering limiting slider with the end of the second rod portion of the first right-angle reversing lever remote from the first rod portion, and a second rotary connection assembly connecting the fourth steering limiting slider with the end of the second rod portion of the second right-angle reversing lever remote from the first rod portion, the horizontal reciprocating tensile stress-strain detection unit comprising two contact heads, denoted as a first contact head and a second contact head, and further comprising:
a first detection slider slidably disposed on the second steering limiting slide rail;
a first contact head mounting plate for mounting the first contact head arranged along the first direction;
a first tensile force sensor for detecting tensile force data generated by the first contact head along the third direction;
a first detection connecting member fixed on the first detection slider, configured to connect the first contact head mounting plate with one end of the first tensile force sensor;
a first displacement sensor for detecting displacement data of the first contact head, the housing of which is disposed along the third direction;
a first displacement sensor mounting frame arranged beside the first detection connecting member for fixing the housing of the first displacement sensor;
a second detection slider slidably disposed on the second steering limiting slide rail;
a second contact head mounting plate for mounting the second contact head arranged along the first direction;
a second tensile force sensor for detecting tensile force data generated by the second contact head along the third direction;
a second detection connecting member fixed on the second detection slider, configured to connect the second contact head mounting plate with one end of the second tensile force sensor;
a second displacement sensor for detecting displacement data of the second contact head, the housing of which is disposed along the third direction; and
a second displacement sensor mounting frame arranged beside the second detection connecting member for fixing the housing of the second displacement sensor,
wherein the other end of the first tensile force sensor is fixedly connected with the second steering limiting slider, the movable core of the first displacement sensor is connected with the first detection connecting member, the other end of the second tensile force sensor is fixedly connected with the fourth steering limiting slider, and the movable core of the second displacement sensor is connected with the second detection connecting member, the first and second contact heads being parallel and located at the same height.

6. The device of claim 4, wherein the reversing unit further comprises a first connection plate fixedly connected to the end of the first rod portion of the first right-angle reversing lever remote from the second rod portion, and a second connection plate fixedly connected to the end of the first rod portion of the second right-angle reversing lever remote from the second rod portion;
wherein the driving unit further comprises a driving connection assembly, the driving connection assembly including a connecting shaft and, sequentially mounted on the connecting shaft, a first fastener, a first driving connector, a first positioning shaft sleeve, a first bearing cover, a first bearing, a shaft collar, a second bearing, a second bearing cover, a second positioning shaft sleeve, a second driving connector, and a second fastener;
wherein the first connection plate is provided with a through hole for embedding the first bearing, and the second connection plate is provided with a through hole for embedding the second bearing;
wherein one end of the first driving connector is provided with a through hole for mounting on the connecting shaft, and the other end of the first driving connector is connected to the output shaft; and
wherein one end of the second driving connector is provided with a through hole for mounting on the connecting shaft, and the other end of the second driving connector is connected to the output shaft.

7. The device of claim 6, wherein the driving unit further comprises:
a driving limiting slide rail extending along the first direction and disposed below the output shaft;
a driving limiting slider slidably disposed on the driving limiting slide rail;
an L-shaped driving connection plate fixed to the driving limiting slider and configured to connect the output shaft with the other ends of the first and second driving connectors; and
a fixed bracket fixedly connected to the bottom of the housing of the driving motor.

8. The device of claim 6, wherein the first fastener and the second fastener are locking nuts, or double nuts, or, based on a single nut, further secured with a double-ear lock washer, a Nord-Lock washer, or a thread locking adhesive.

9. The device of claim 2, wherein the vertical effective leg lengths of the first rod portion and the second rod portion of the right-angle reversing lever are a first preset length and a second preset length, respectively, such that, by means of the first preset length, the second preset length, and the included angle, the displacement of the contact head is in a fixed geometric ratio relationship with the displacement of the output shaft.

10. The device of claim 9, wherein the included angle is 45 degrees, and the first preset length and the second preset length are equal, such that the displacement of the contact head equals the displacement of the output shaft.
